(19) United States
(12) Patent Application Publication
(10) Pub. No.: US 2025/0360233 A1
(43) Pub. Date: Nov. 27, 2025

(54) Central Neurodegeneration Imaging Agent and Method of Preparing the Same

(71) Applicant: National Atomic Research Institute, Taoyuan (TW)

(72) Inventors: Shiou-Shiow Farn, Taoyuan (TW); Hung-Man Yu, Taoyuan (TW); Tsung-Yu Shih, Nantou (TW); Kuan-Yin Chen, Taoyuan (TW); Wei-Hsi Chen, Taoyuan (TW)

(21) Appl. No.: 18/885,690

(22) Filed: Sep. 15, 2024

(30) Foreign Application Priority Data
May 24, 2024 (TW) 113119416

(51) Int. Cl. A61K 51/04

(52) U.S. Cl. CPC A61K 51/0446; A61K 2123/00

(57) ABSTRACT

A central neurodegeneration imaging agent and a method of preparing the same include steps of a reaction between a central nerve positron imaging agent precursor and a marker Fluorine-18 (F-18) and an imaging agent formula. After the labeling process, F-18 molecules specifically bind to misfolded α-synuclein to display lesions in images of positron emission tomography (PET). Therefore, in animal mode brain imaging images, the invention indicates that the F-18-α-syn3 imaging agent achieves an obviously better images of the brains of rotenone-treated mice with Parkinson's disease (PD) than mice not treated with rotenone in experiments.

### FIELD OF THE INVENTION

The present invention relates to a central neurodegeneration imaging agent and a method of preparing the same. More particularly, the present invention relates to a reagent formula and process criteria required for a Fluorine-18 (F-18) to undergo a labeling reaction with an imaging agent precursor. More particularly, after the labeling process, F-18 molecules specifically bind to misfolded α-synuclein (α-synuclein) to display lesions in images of positron emission tomography (PET).

### DESCRIPTION OF THE PRIOR ART

It is not easy for central neurological drugs to pass through the blood-brain barrier (BBB). In view of this, a foreign paper (Wenhua Chu et al.) proposes that, in a series of ligands, compound 46a manifests the highest (ki=2 nM) affinity to α-synuclein fibrils, surpasses amyloid-β (Aβ) and Tau proteins (142.4 nM and 80.1 nM) in affinity to α-synuclein fibrils, and exhibits specificity. However, owing to its high lipophilicity and high log P value (estimated at 4.7 by ChemBioDraw and at 4.18 by experiments), the compound is disadvantaged by a high degree of background binding (imaging results reveal high non-specific noise); thus, Wenhua Chu et al. asserts that, owing to its high Log P value, compound 46a is unlikely to bind with α-synuclein in the brain lesions of patients with Parkinson's disease (PD) reliably and constantly. Therefore, Wenhua Chu et al. asserts that F-18 46a is not suitable for functioning as a PET imaging agent and proposes imaging Lewy bodies (LB) through quantifying the accumulation of α-synuclein in the brain. Therefore, it is imperative to not only address the issues with the passage of drugs through the blood-brain barrier (BBB) but also overcome the aforesaid drawbacks of the prior art.

### BRIEF SUMMARY OF THE INVENTION

Therefore, the main purpose of the present invention is to overcome the aforesaid drawbacks of the prior art and provide a central neurodegeneration imaging agent and a method of preparing the same, and especially a reagent formula and process criteria required for an F-18 to undergo a labeling reaction with an imaging agent precursor such that, after the labeling process, F-18 molecules specifically bind to misfolded α-synuclein to display lesions in images of positron emission tomography (PET).

Another purpose of the present invention is to provide a central neurodegeneration imaging agent and a method of preparing the same which not only effectively reduce the logP value but also pass a test of affinity, specificity, and parallel artificial membrane.

To achieve the above purposes, the present invention is a central neurodegeneration imaging agent and a method of preparing the same, comprising F-18 solution (F-18 solution) preparation step, F-18-α-syn3 solution preparation step, and F-18-α-syn3 solution formula step sequentially, wherein:

the F-18 solution preparation step comprises:

step 1: introducing an F-18 solution into a first cartridge, withdrawing an elution solution for rinsing the first cartridge, collecting the F-18 solution rinsed out by a first eppendorf, and measuring activity of the F-18 solution;

step 2: repeating the rinsing of step 1 twice, collecting the F-18 solutions rinsed out by a second eppendorf and a third eppendorf, and measuring activities of the F-18 solutions respectively;

step 3: withdrawing the F-18 solutions of the first eppendorf, the second eppendorf and the third eppendorf according to operation-required activity, introducing the F-18 solutions withdrawn into a first container, measuring the activities of the F-18 solutions in the first container, heating the first container until the first container is totally dry and free of the F-18 solutions;

step 4: introducing an organic solvent into the first container, heating the first container until the first container is totally dry, and repeating step 4 three times;

the F-18-α-syn3 solution preparation step comprises:

step 5: introducing a precursor into the first container, blending the precursor therein, heating the first container, and measuring activity of the precursor therein;

step 6: taking a tiny amount of a crude product out of the first container by suction, dropping the tiny amount of the crude product onto a first thin-layer chromatography (TLC) plate, inserting the first TLC plate vertically into an expansion slot containing an expansion solution to perform instant thin-layer chromatography (iTLC), and calculating labeling efficiency;

step 7: introducing totally a crude product of an F-18-α-syn3 precursor solution obtained by heating the first container into an activated silica column and measuring residual activity of the first container;

step 8: withdrawing an organic solvent for rinsing the silica column, collecting a plurality of rinsing solutions, and measuring the activity of the silica column and each of the rinsing solutions;

step 9: taking a tiny amount of an end product with maximum activity out of the plurality of rinsing solutions of step 8 by suction, dropping the tiny amount of the end product onto a second TLC plate, inserting the second TLC plate vertically into an expansion slot containing an expansion solution to perform iTLC analysis, and calculating labeling efficiency;

step 10: withdrawing the end products of the plurality of rinsing solutions with maximum activity respectively, introducing the end products withdrawn into a second container, heating and evaporating the end products in the second container to concentrate the end product, causing the end product to become 1 mL in volume;

step 11: withdrawing double-distilled water (ddH2O), introducing the double-distilled water withdrawn into the second container, measuring the activity of the double-distilled water in the second container, separating and purifying the end product with high-performance liquid chromatography (HPLC);

step 12: collecting first and second separated-peak solution (having activity) test tubes separately according to the HPLC separated-peak retention time and activity, and measuring their activities respectively;

step 13: withdrawing and introducing the ddH2O into the second separated-peak solution test tube to perform dilution and measuring the activity of the ddH2O therein;

the F-18-α-syn3 solution formula step comprises:

step 14: withdrawing and introducing the separated, purified and diluted solution of step 13 into an activated second cartridge and measuring radioactivity of the second cartridge;

step 15: withdrawing an organic solvent for rinsing the second cartridge, collecting a plurality of rinsing solutions, and measuring the activities of the second cartridge and each of the rinsing solutions;

step 16: selecting, taking by suction, and introducing the rinsing solution with maximum activity of step 15 into a first eppendorf and measuring its activity;

step 17: sequentially taking by suction and introducing a surfactant, an organic solvent and a normal saline into the first eppendorf, blending the surfactant, the organic solvent and the normal saline to become an F-18-α-syn3 formula solution, observing a color of the F-18-α-syn3 formula solution, and measuring its activity; and

step 18: withdrawing the F-18-α-syn3 formula solution, filtering the F-18-α-syn3 formula solution with a microporous membrane, collecting and introducing an F-18-α-syn3 labeling finished product solution into the second eppendorf, and measuring its activity to prepare an imaging agent F-18-α-syn3.

In the aforesaid embodiment of the present invention, the elution solution is a solution obtained by dissolving Kryptofix 2.2.2. and potassium carbonate (K₂CO₃) in an organic solvent.

In the aforesaid embodiment of the present invention, the first container in step 3 is a hermetically sealed container filled with nitrogen gas (N₂ gas), placed on a heating plate, and heated at a temperature above 100° C.

In the aforesaid embodiment of the present invention, a precursor preparation step takes place between the F-18 solution preparation step and the F-18-α-syn3 solution preparation step and entails dissolving the precursor of step 5 in an organic solvent solution, introducing a resultant solution into the first container, and blending the resultant solution.

In the aforesaid embodiment of the present invention, the first container of step 5 is placed on a heating plate and heated for 8˜12 minutes.

In the aforesaid embodiment of the present invention, the first container and the second container are vials.

In the aforesaid embodiment of the present invention, an organic solvent is introduced into the silica column and left to stand or its removal therefrom is speeded up by air in order to be activated.

In the aforesaid embodiment of the present invention, an organic solvent and ddH₂O are sequentially introduced into the second cartridge and left to stand or their removal therefrom is speeded up by air in order to be activated.

In the aforesaid embodiment of the present invention, the microporous membrane is a polymeric membrane made from polyethylene, polypropylene, polystyrene, poly (methyl methacrylate), polyvinyl chloride, nylon, polycarbonate, polyurethane, polytetrafluoroethylene or polyethylene terephthalate and has a pore diameter of 0.22 μm.

### BRIEF DESCRIPTION OF THE DRAWINGS

FIG. 1 is a schematic view of the process flow of a method of preparing a central neurodegeneration imaging agent according to the present invention.

FIG. 2A is a schematic view of performing brain imaging with F-18-FE-PE21 and comparing the intake of an F-18 imaging agent into a mouse brain according to the present invention.

FIG. 2B is a schematic view of performing brain imaging with F-18-FDOPA and comparing the intake of an F-18 imaging agent into a mouse brain according to the present invention.

FIG. 2C is a schematic view of performing brain imaging with F-18-α-syn3 and comparing the intake of an F-18 imaging agent into a mouse brain according to the present invention.

### DESCRIPTION OF THE PREFERRED EMBODIMENTS

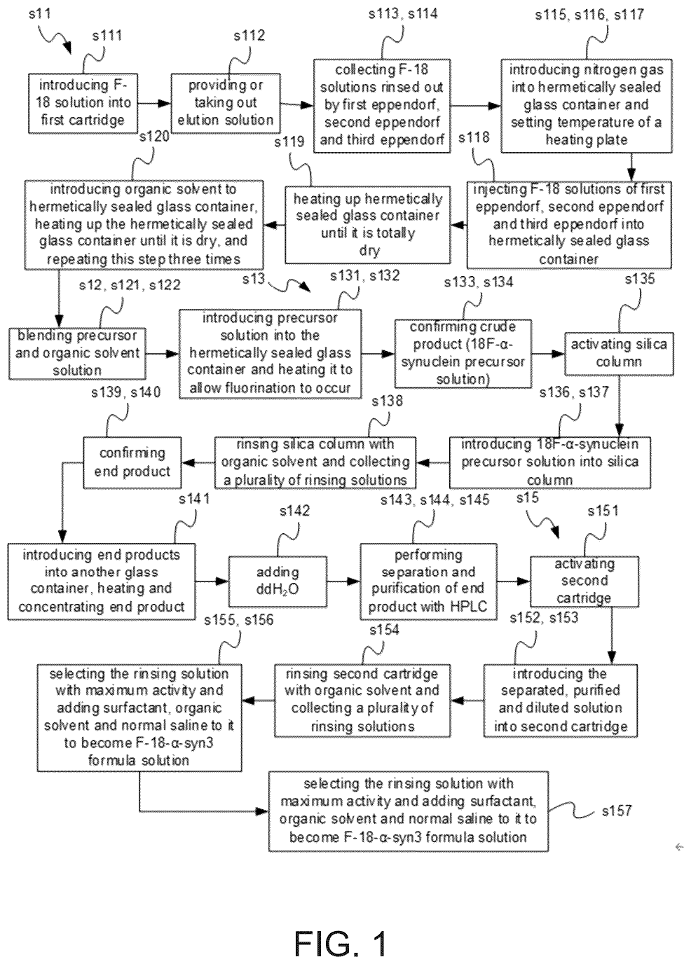

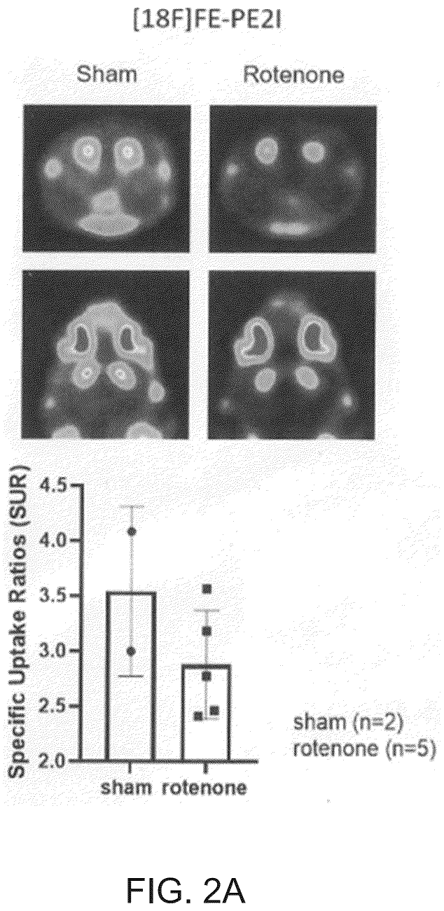

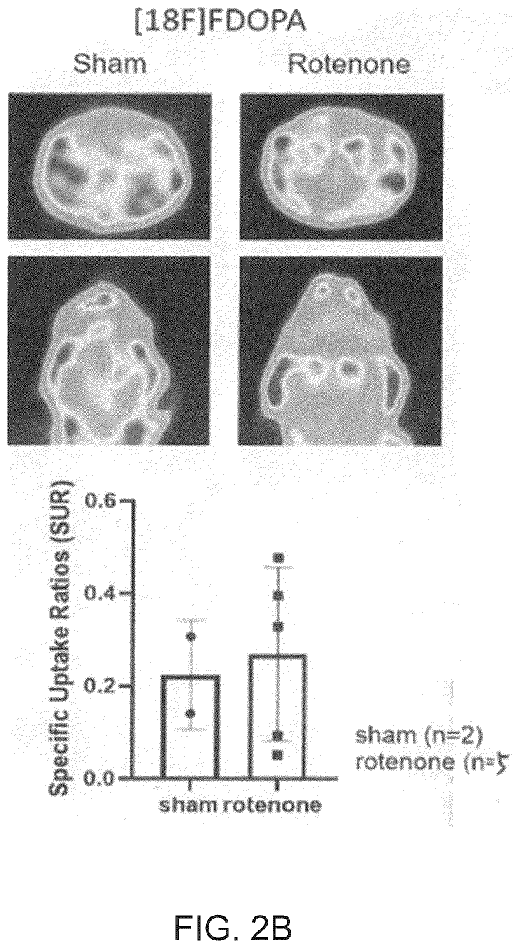

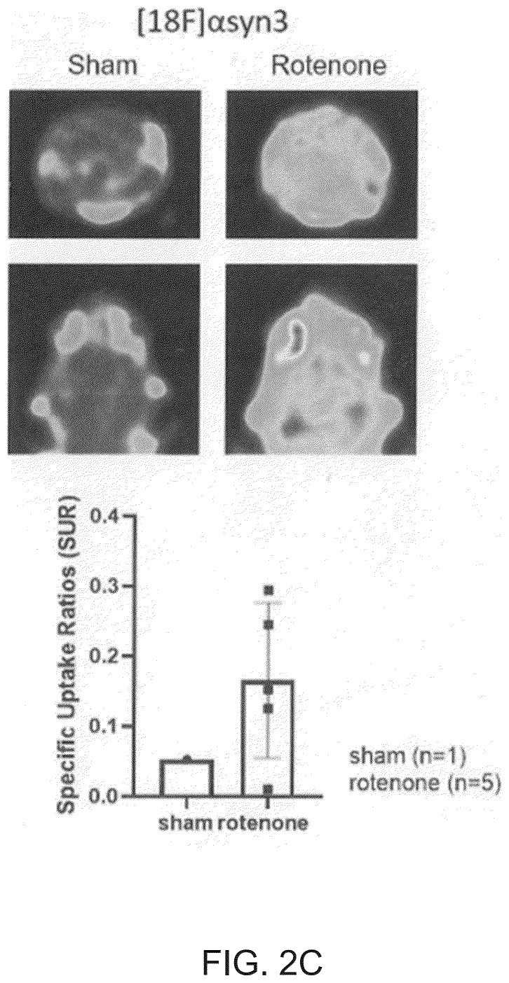

Referring to FIG. 1˜FIG. 2C, there are shown a schematic view of the process flow of a method of preparing a central neurodegeneration imaging agent according to the present invention, a schematic view of performing brain imaging with F-18-FE-PE21 and comparing the intake of an F-18 imaging agent into a mouse brain according to the present invention, a schematic view of performing brain imaging with F-18-FDOPA and comparing the intake of an F-18 imaging agent into a mouse brain according to the present invention, and a schematic view of performing brain imaging with F-18-α-syn3 and comparing the intake of an F-18 imaging agent into a mouse brain according to the present invention respectively. As shown in the diagrams, the present invention is a central neurodegeneration imaging agent and a method of preparing the same, comprising Fluorine-18 solution (F-18 solution) preparation step s11, precursor preparation step s12, F-18-α-syn3 solution preparation step s13, and F-18-α-syn3 solution formula step s15 sequentially.

The F-18 solution preparation step s11 comprises: step s111: taking a first cartridge and placing a first eppendorf under the first cartridge; opening a hermatically sealed first container, withdrawing an F-18 solution therefrom, and introducing the F-18 solution into the first cartridge slowly; collecting a rinsing solution in the first eppendorf and discarding it;

step s112: providing or taking out an elution solution, wherein the elution solution is a solution obtained by dissolving Kryptofix 2.2.2. and potassium carbonate (K₂CO₃) in an organic solvent (for example, absolute acetonitrile (ACN));

step s113: withdrawing the elution solution with a syringe, rinsing the first cartridge of step s111, collecting the F-18 solution rinsed out by the first eppendorf, and measuring its activity;

step s114: repeating step s113 twice, collecting the F-18 solutions rinsed out by a second eppendorf and a third eppendorf, and measuring their activities respectively;

step s115: closing a second container with a plastic cork and then hermetically sealing the second container; step s116: inserting another syringe containing therein cotton and activated carbon into the second container of step s115; inserting a nitrogen gas (N₂ gas) needle into the hermetically sealed second container, introducing nitrogen gas into the second container, and confirming that the nitrogen gas is movable smoothly;

step s117: setting the temperature of the heating plate to be higher than 100° C.;

step s118: taking the F-18 solution of steps s113˜s114 with a syringe (according to operation-required activity), injecting the F-18 solution into the hermetically sealed second container of step s116, and measuring its activity;

step s119: placing the hermetically sealed second container of step s118 on the heating plate, heating up the hermetically sealed second container until the hermetically sealed second container is totally dry, and confirming that the hermetically sealed second container is free of any solution; and

step s120: introducing an organic solvent to the hermetically sealed second container of step s119, heating up the second container until it is totally dry, and repeating this step three times.

The precursor preparation step s12 comprises:

step s121: weighing an appropriate amount of a precursor with a scale and placing the precursor in a second eppendorf;

step s122: withdrawing an organic solvent solution with a syringe, introducing the organic solvent solution into the second eppendorf of step s121, vibrating and blending the organic solvent solution therein for subsequent use;

The F-18-α-syn3 solution preparation step s13 comprises:

step s131: withdrawing, with a syringe, the precursor completely dissolved in step s122, introducing the precursor withdrawn into the hermetically sealed second container of step s120, and blending the precursor therein;

step s132: placing the hermetically sealed second container of step s131 on a heating plate, heating the heating plate for 10 minutes, observing and timing the color and activity of the reacting solution;

step s133: providing a standard first thin-layer chromatography (TLC) plate 5 cm in length and an organic solvent expansion slot, taking a tiny amount of the crude product of step s132 through capillary action, dropping the tiny amount of the crude product onto the first TLC plate at the point of 1 cm in length, and inserting the plate vertically into the expansion slot to perform chromatography;

step s134: performing instant thin-layer chromatography (iTLC) and calculating labeling efficiency;

step s135: providing a silica column, placing a third eppendorf under the silica column, withdrawing an appropriate volume of an organic solvent with a syringe, introducing the organic solvent into the silica column slowly, leaving the organic solvent therein to stand or speeding up the removal of the organic solvent by air, collecting a rinsing solution in the third eppendorf, discarding it, and placing a fourth eppendorf under the activated silica column;

step s136: opening the hermetically sealed second container of step s132, taking, with a syringe, a crude product of an F-18-α-syn3 precursor solution obtained by heating, introducing the crude product into the activated silica column of step s135, withdrawing the crude product totally, collecting a rinsing solution in the fourth eppendorf, and discarding it;

step s137: measuring residual activity of the second container;

step s138: taking an organic solvent with a syringe, introducing the organic solvent into the silica column of the rinsing step s136, collecting a plurality of rinsing solutions (and numbering them with the numbers 1, 2, . . . ), measuring the activities of the silica column and each of the rinsing solutions, and confirming the completion of the collection;

step s139: providing a standard second TLC plate 5 cm in length and an organic solvent expansion slot, taking a tiny amount of the end product with maximum activity of step s138 through capillary action, dropping the tiny amount of the end product onto the second TLC plate at the point of 1 cm in length, inserting the plate vertically into the expansion slot, and performing chromatography;

step s140: performing iTLC analysis and calculating labeling efficiency;

step s141: selecting the end products with maximum activity totaling to an extent sufficient for use in subsequent experiments to take the selected end products with a syringe and number them with the numbers 1, 2, . . . , introducing the end products thus taken into a third container, heating and evaporating the end products in the third container to concentrate the end product, causing the end product to become 1 mL in volume (or be supplemented with an organic solvent when less than 1 mL);

step s142: withdrawing ddH2O with a syringe, introducing the ddH2O into the third container of step s141, and measuring its activity;

step s143: performing the separation and purification of the end product with high-performance liquid chromatography (HPLC);

step s144: providing two new test tubes, collecting the first and second separated-peak solutions (each having activity) separately according to HPLC separated-peak retention time and activity, and measuring their activities; and step s145: withdrawing ddH₂O with a syringe, introducing the ddH₂O into the second separated-peak solution test tube of step s144 to undergo dilution, and measuring its activity.

The F-18-α-syn3 solution formula step s15 comprises:

step s151: providing a second cartridge, placing the fifth eppendorf under the second cartridge, withdrawing an appropriate amount of an organic solvent with a syringe, introducing the appropriate amount of the organic solvent into the second cartridge slowly, withdrawing an appropriate amount of ddH₂O with a syringe, introducing the appropriate amount of ddH₂O into the second cartridge slowly, leaving the ddH₂O therein to stand or speeding up the removal of the solution of the organic solvent and the ddH₂O by air, discarding the solution, finishing the activation of the second cartridge (100 mg);

step s152: taking, with a 1 ml syringe, the separated, purified and diluted solution of step s145, introducing the solution into the activated second cartridge of step s151, collecting a rinsing solution, and discarding it;

step s153: measuring the second cartridge radioactivity;

step s154: withdrawing an organic solvent with a syringe, collecting a plurality of rinsing solutions (being equal in volume and being numbered) with the second cartridge of the rinsing step s152, and measuring the activities of the second cartridge and each of the rinsing solutions;

step s155: selecting, from the plurality of rinsing solutions of step s154, the one with maximum activity, taking and introducing an appropriate volume of the selected rinsing solution into a sixth eppendorf with a microabsorber, and measuring its activity;

step s156: taking by suction an appropriate volume of a surfactant, organic solvent and normal saline sequentially, introducing them into the sixth eppendorf of step s155, blending them therein gently to become an F-18-α-syn3 formula solution, observing the color of the F-18-α-syn3 formula solution, and measuring its activity; and

step s157: withdrawing the F-18-α-syn3 formula solution of step s156 with a syringe, mounting a microporous membrane (with a diameter of 13 mm and a pore diameter of 0.22 μm) on the front end of the syringe, filtering and collecting an F-18-α-syn3 labeling finished product solution with the second eppendorf, and measuring its activity. At this point in time, the preparing of the imaging agent F-18-α-syn3 is finished. The F-18-α-syn3 is (Z)-1-(2-(2-fluoroethoxy) ethyl)-3-((E)-3-(4-nitrophenyl)allyl idene) indolin-2-one, with a molecular formula C₂₁H₁₉FN₂O₄, and a molecular weight of 382.1. The F-18-α-syn3 has a structural formula shown below:

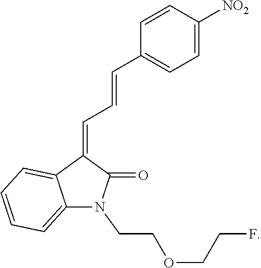 [SMILES: O=C1/C(=C\C=C\c2ccc([N+](=O)[O-])cc2)c2ccccc2N1CCOCCF]

Therefore, the present invention is a novel central neurodegeneration imaging agent and a method of preparing the same.

In a preferred, specific embodiment of the present invention, the first container, the second container and the third container are vials.

In a preferred, specific embodiment of the present invention, the microporous membrane is a polymeric membrane made from polyethylene, polypropylene, polystyrene, poly(methyl methacrylate), polyvinyl chloride, nylon, polycarbonate, polyurethane, polytetrafluoroethylene or polyethylene terephthalate.

In a preferred, specific embodiment of the present invention, the precursor has a structural formula shown below:

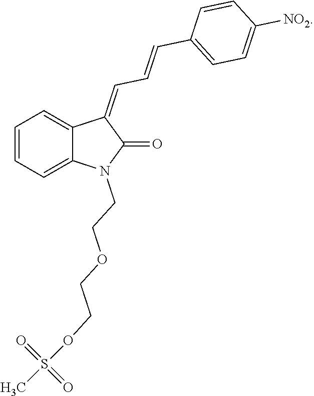 [SMILES: CS(=O)(=O)OCCOCCN1C(=O)/C(=C\C=C\c2ccc([N+](=O)[O-])cc2)c2ccccc21]

The embodiment described below is provided for exemplary purposes to explain the details and technical features of the present invention but is not restrictive of the claims of the present invention.

Regarding its application, the present invention entails feeding Rotenone to mice by tube feeding continuously for 12 weeks to induce the mouse brains to manifest Parkinson's disease (PD) symptom animal mode for function as the experimental group, treating mice with placebo to serve as the control group (Sham) without manifesting any PD lesions, performing imaging of the mouse brains with an F-18-FE-PE21, F-18-FDOPA and F-18-α-syn3 respectively, displaying the longitudinal-section and cross-section images of the mouse brains, and comparing the intake of the F-18 imaging agent into the mouse brains. The analysis results are shown in FIG. 2A˜FIG. 2C. FIG. 2C shows that, compared with the control group, the present invention provides clearer F-18 images of the F-18-α-syn3 and an obviously lower background absorption level to discern PD brain lesions. FIG. 2A and FIG. 2B show no difference and no specificity in PD brain tissues between the F-18-FE-PE21 and the F-18-FDOPA. Further findings of FIG. 2A and FIG. 2B are as follows: the experimental group has lower F-18-FE-PE21 intake than the control group, whereas F-18-FDOPA exhibits no selectivity in terms of brain distribution.

The experimental results of the logP analysis of the F-18-α-syn3 of the present invention are as follows:

F-18-α-syn3 (50 uCi), logP=2.63 (SD=0.56);

F-18-α-syn-3 (end solution, 22 uCi), logP=2.41 (SD=0.5); and

F-18-α-syn-3 (end solution, 50 uCi), logP=2.07 (SD=0.22).

The above data is about testing the logP of the F-18-α-syn3 labeling finished product solution repeatedly. The result confirms that all the logP values range from 2 to 3. Therefore, animal brain imaging indicates that protein accumulation and imaging effect occur to α-synuclein.

Moreover, the present invention discloses giving considerations to the F-18-α-syn3 formula and serum stability and thus conducting F-18-α-syn3 formula research to attain a recycling rate as high as 90.1%. Regarding a test of F-18-α-syn3 serum stability, medications are placed within serum at 37° C., achieving radioactive chemical purity of 91.5% even after a hour.

The imaging agent molecular formula F-18-α-syn3 disclosed by the present invention is obtained by making a change to a conventional imaging agent (F-18 46a), with a LogP of 3.12 (estimated at 3.12 by software, with an experimental value ranging from 2.07 to 2.63 as mentioned above), i.e., lower than the conventional imaging agent F-18 46a (with a LogP value of 4.18 as calculated by software), not to mention that the structural effectiveness of F-18-α-syn3 is confirmed by animal mode images. Experimental results indicate that the F-18-α-syn3 of the present invention exhibits high affinity (ki) to α-synuclein. A test tube competition test reveals an IC50 (50% binding inhibition) of 172 nM that has reached nM scale, way better than amyloid-β(Aβ) and Tau proteins (142 nM and 80 nM), and high specificity. As revealed by the parallel artificial membrane permeation assay (PAMPA) that simulates the passage of drugs through the blood-brain barrier (BBB), a concentration of at least 6 nM ensures that the satisfaction of PAMPA, and a concentration of 60 nM achieves a pass rate of 56.25%. The aforesaid test of the effect of the F-18-α-syn3 on rodent-gene mice also proves that the F-18-α-syn3 can pass through the blood-brain barrier (BBB) to enter mouse brains. Therefore, the present invention not only effectively reduces the logP value but also passes a test of affinity, specificity, and parallel artificial membrane.

In conclusion, the present invention is a central neurodegeneration imaging agent and a method of preparing the same, effective in overcoming the drawbacks of the prior art, providing the F-18-α-syn3 that not only effectively reduces the logP value but also passes a test of affinity, specificity, and parallel artificial membrane and thus is suitable for use as a imaging agent, enabling the present invention to be inventive and practical and thereby meet patentability requirements.

The preferred embodiments herein disclosed are not intended to unnecessarily limit the scope of the invention. Therefore, simple modifications or variations belonging to the equivalent of the scope of the claims and the instructions disclosed herein for a patent are all within the scope of the present invention.

What is claimed is:

1. A method of preparing a central neurodegeneration imaging agent, comprising Fluorine-18 solution (F-18 solution) preparation step, F-18-α-syn3 solution preparation step, and F-18-α-syn3 solution formula step sequentially, wherein:
the F-18 solution preparation step comprises:
step 1: introducing an F-18 solution into a first cartridge, withdrawing an elution solution for rinsing the first cartridge, collecting the F-18 solution rinsed out by a first eppendorf, and measuring activity of the F-18 solution;
step 2: repeating the rinsing of step 1 twice, collecting the F-18 solutions rinsed out by a second eppendorf and a third eppendorf, and measuring activities of the F-18 solutions respectively;
step 3: withdrawing the F-18 solutions of the first eppendorf, the second eppendorf and the third eppendorf according to operation-required activity, introducing the F-18 solutions withdrawn into a first container, measuring the activities of the F-18 solutions in the first container, heating the first container until the first container is totally dry and free of the F-18 solutions;
step 4: introducing an organic solvent into the first container, heating the first container until the first container is totally dry, and repeating step 4 three times;
the F-18-α-syn3 solution preparation step comprises:
step 5: introducing a precursor into the first container, blending the precursor therein, heating the first container, and measuring activity of the precursor therein;
step 6: taking a tiny amount of a crude product out of the first container by suction, dropping the tiny amount of the crude product onto a first thin-layer chromatography (TLC) plate, inserting the first TLC plate vertically into an expansion slot containing an expansion solution to perform instant thin-layer chromatography (iTLC), and calculating labeling efficiency;
step 7: introducing totally a crude product of an F-18-α-syn3 precursor solution obtained by heating the first container into an activated silica column and measuring residual activity of the first container;
step 8: withdrawing an organic solvent for rinsing the silica column, collecting a plurality of rinsing solutions, and measuring the activity of the silica column and each of the rinsing solutions;
step 9: taking a tiny amount of an end product with maximum activity out of the plurality of rinsing solutions of step 8 by suction, dropping the tiny amount of the end product onto a second TLC plate, inserting the second TLC plate vertically into an expansion slot containing an expansion solution to perform iTLC analysis, and calculating labeling efficiency;
step 10: withdrawing the end products of the plurality of rinsing solutions with maximum activity respectively, introducing the end products withdrawn into a second container, heating and evaporating the end products in the second container to concentrate the end product, causing the end product to become 1 mL in volume;
step 11: withdrawing ddH2O, introducing the double-distilled water withdrawn into the second container, measuring the activity of the double-distilled water in the second container, separating and purifying the end product with high-performance liquid chromatography (HPLC);
step 12: collecting first and second separated-peak solution (having activity) test tubes separately according to the HPLC separated-peak retention time and activity, and measuring their activities respectively;
step 13: withdrawing and introducing the ddH₂O into the second separated-peak solution test tube to perform dilution and measuring the activity of the ddH₂O therein;
the F-18-α-syn3 solution formula step comprises:
step 14: withdrawing and introducing the separated, purified and diluted solution of step 13 into an activated second cartridge and measuring radioactivity of the second cartridge;
step 15: withdrawing an organic solvent for rinsing the second cartridge, collecting a plurality of rinsing solutions, and measuring the activities of the second cartridge and each of the rinsing solutions;
step 16: selecting, taking by suction, and introducing the rinsing solution with maximum activity of step 15 into a first eppendorf and measuring its activity;
step 17: sequentially taking by suction and introducing a surfactant, an organic solvent and a normal saline into the first eppendorf, blending the surfactant, the organic solvent and the normal saline to become an F-18-α-syn3 formula solution, observing a color of the F-18-α-syn3 formula a solution, and measuring its activity; and
step 18: withdrawing the F-18-α-syn3 formula solution, filtering the F-18-α-syn3 formula solution with a microporous membrane, collecting and introducing an F-18-α-syn3 labeling finished product solution into the second eppendorf, and measuring its activity to prepare a imaging agent F-18-α-syn3, wherein the F-18-α-syn3 is (Z)-1-(2-(2-fluoroethoxy) ethyl)-3-((E)-3-(4-nitrophenyl) a llylidene) indolin-2-one, and the F-18-α-syn3 has a structural formula shown below:

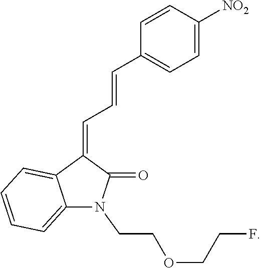 [SMILES: O=C1/C(=C\C=C\c2ccc([N+](=O)[O-])cc2)c2ccccc2N1CCOCCF]

2. The method of claim 1, wherein the elution solution is a solution obtained by dissolving Kryptofix 2.2.2. and potassium carbonate (K₂CO₃) in an organic solvent.

3. The method of claim 1, wherein the first container in step 3 is a hermetically sealed container filled with nitrogen gas (N₂ gas), placed on a heating plate, and heated at a temperature above 100° C.

4. The method of claim 1, wherein a precursor preparation step takes place between the F-18 solution preparation step and the F-18-α-syn3 solution preparation step and entails dissolving the precursor of step 5 in an organic solvent solution, introducing a resultant solution into the first container, and blending the resultant solution.

5. The method of claim 1, wherein the first container of step 5 is placed on a heating plate and heated for 8˜12 minutes.

6. The method of claim 1, wherein the first container and the second container are vials.

7. The method of claim 1, wherein an organic solvent is introduced into the silica column and left to stand or its removal therefrom is speeded up by air in order to be activated.

8. The method of claim 1, wherein an organic solvent and ddH₂O are sequentially introduced into the second cartridge and left to stand or their removal therefrom is speeded up by air in order to be activated.

9. The method of claim 1, wherein the microporous membrane is a polymeric membrane made from polyethylene, polypropylene, polystyrene, poly (methyl methacrylate), polyvinyl chloride, nylon, polycarbonate, polyurethane, polytetrafluoroethylene or polyethylene terephthalate and has a pore diameter of 0.22 μm.

10. The method of claim 1, wherein the precursor has a structural formula shown below: 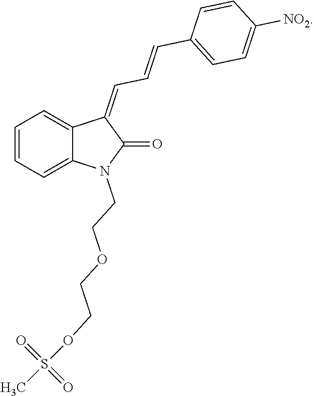 [SMILES: CS(=O)(=O)OCCOCCN1C(=O)/C(=C\C=C\c2ccc([N+](=O)[O-])cc2)c2ccccc21]
